(19) United States
(12) Patent Application Publication
(10) Pub. No.: US 2026/0166124 A1
(43) Pub. Date: Jun. 18, 2026

(54) INTRANASAL ADMINISTRATION OF NGF FOR THE PREVENTION OR TREATMENT OF RETINOPATHIES AND OPTIC NERVE DISORDERS

(71) Applicant: Dompe` farmaceutici S.P.A., Milano (IT)

(72) Inventors: Marcello ALLEGRETTI, L`Aquila (IT); Franca CATTANI, L`Aquila (IT); Maria Irene ANTONANGELI, L`Aquila (IT); Andrea ARAMINI, L`Aquila (IT); Rubina NOVELLI, Milan (IT)

(21) Appl. No.: 19/423,385

(22) Filed: Dec. 17, 2025

(30) Foreign Application Priority Data
Dec. 17, 2024 (EP) 24220746.2
Jul. 30, 2025 (EP) 25192962.6

(51) Int. Cl. A61K 38/18; A61K 9/00; A61P 27/02

(52) U.S. Cl. CPC A61K 38/185; A61K 9/0043; A61P 27/02

(57) ABSTRACT

The present invention relates to Nerve growth factor (NGF) or a mutein thereof for use in the prevention or treatment of a retinopathy or of an optic nerve disorder in a subject, wherein said NGF is administered intranasally to the subject.

### CROSS-REFERENCE TO RELATED APPLICATIONS

This application claims priority to European Patent Application No. 24220746.2, filed Dec. 17, 2025, and European Patent Application No. 25192962.6, filed Jul. 30, 2025. The contents of these applications are incorporated herein by reference in their entireties.

### SEQUENCE LISTING

This application contains a Sequence Listing that has been submitted electronically as an XML file named “36381-0065001_SL_ST26.XML.” The XML file, created on Dec. 17, 2025, is 7,007 bytes in size. The material in the XML file is hereby incorporated by reference in its entirety.

### FIELD OF THE INVENTION

The present invention relates to NGF for use in the prevention or treatment of a retinopathy or of an optic nerve disorder in a subject, wherein said NGF is administered intranasally to the subject.

The present invention further relates to a mutein of NGF for use in the prevention or treatment of a retinopathy or of an optic nerve disorder in a subject, wherein said mutein of NGF is administered intranasally to the subject.

### STATE OF THE ART

The nerve growth factor (NGF) is a member of the family of evolutionarily well-conserved neurotrophin growth factors that are required for the development and survival of specific neuronal populations, which also includes brain-derived neurotrophic factor (BDNF), neurotrophin-3 (NT3) and NT4/5. The NGF sequence is well preserved among different species, with 90% homology between murine and human NGF.

NGF and its receptors are expressed both in the retina and in the optic nerve. Specifically, NGF is expressed by retinal pigment epithelium, Müller cells, and retinal ganglion cells. NGF receptors are found on retinal pigment epithelium, photoreceptors, Müller cells, retinal ganglion cells, in the visual cortex and in oligodendrocytes of the optic nerve (Carmignoto G, et al., Exp Neurol. 1991; 111 (3): 302-311; Chakrabarti S, et al., Brain Res. 1990; 523 (1): 11-15; Lambiase A, et al., Invest Ophthalmol Vis Sci. 1998; 39:1272-1275; Rossi F M et al., J Neurosci. 2002; 22 (3): 912-919; Cohen R I et al., J Neurosci. 1996; 16 (20): 6433-6442).

There is both preclinical and clinical evidence in the literature of the therapeutic potential of NGF for the treatment of retinopathies and optic nerve disorders (Kanu et al., Semin Ophthalmol. 2021 May 19; 36 (4): 224-231).

Preclinical studies demonstrated that the administration of NGF is effective in several pathological settings involving the retina and the optic nerve, including optic nerve transection (Carmignoto G. et al., J Neurosci Off J Soc Neurosci. 1989; 9 (4): 1263-1272), retinal detachment (Xiaodong Sun et al., Ophthalmologica 2008; 222 (1): 58-61), phototoxic retinopathy (Rocco et al., Graefes Arch Clin Exp Ophthalmol 2018, 256:729-738), retinal ischemia (Siliprandi R. et al., Invest Ophthalmol Vis Sci. 1993; 34:3232-3245; Chen et al., Growth Factors 2015; 33 (2): 149-159), glaucoma (Colafrancesco V. et al., J Glaucoma. 2011; 20 (2): 100-108; Lambiase A. et al., Proc Natl Acad Sci USA. 2009; 106 (32): 13469-13474; Lambiase A. et al., Graefes Arch Clin Exp Ophthalmol Albrecht Von Graefes Arch Klin Exp Ophthalmol. 1997; 235 (12): 780-785), diabetic retinopathy (Hammes H P et al., Mol Med. 1995; 1 (5): 527-534, Mantelli D F. et al., Eur J Ophthalmol Published Online May 2013; 9) and retinal degeneration causing photoreceptor loss (Lenzi L., et al., Vision Res. 2005; 45 (12): 1491-1500; Lambiase A. et al., Graefes Arch Clin Exp Ophthalmol. 1996; 234 (1): S96-S100).

There is also clinical evidence that the administration of NGF is effective in patients with glaucoma (Lambiase A. et al., Proc Natl Acad Sci USA. 2009; 106 (32): 13469-13474), optic pathway gliomas (Falsini B. et al., Neurorehabil Neural Repair. 2011; 25 (6): 512-520; Falsini B. et al., Brain J Neurol. 2016; 139 (Pt 2): 404-414), retinitis pigmentosa (Falsini B. et al., J Transl Med. 2016; 14; 229 (3): 746-753), Non-Arteritic Anterior Ischemic Optic Neuropathy (NAION) (Chen Kai et al., Chin J Optom Ophthalmol Vis Sci, 2020, 22 (10): 781-785), macular hole (Zhang et al., BMC Ophthalmol 2019, 19 (1): 130) and non-neovascular age-related macular degeneration (Lambiase A. et al., Ann Ist Super Sanita. 2009; 45 (4): 439-442). The administration of NGF shows protective effects and mitigates the retinal and optic nerve damage and degeneration induced by these conditions.

The current most used methods of delivering drugs to the eye are topical administration in the form of eye drops or direct injection to internal structures of the eye, such as intravitreal or retrobulbar injection. Typically, repeated administrations are necessary to maintain therapeutically relevant concentrations of the drug at the site of action.

Topical administration is non-invasive; however, it faces the challenge of achieving therapeutic drug concentrations in the retina and optic nerve due rapid drug clearance from the ocular surface and the difficulty of penetrating the inner eye tissues, with only a small fraction of the administered dose reaching the posterior segment of the eye.

The administration of a therapeutic agent directly into the internal structures of the eye may be perceived as invasive, uncomfortable, or even painful by patients, thus limiting their compliance with therapy.

Furthermore, the above-mentioned intravitreal injection is associated with some risks, such as irritation, subconjunctival hemorrhage, increased intraocular pressure (IOP), cataract formation, occurrence of post-injection infections (endophthalmitis), corneal abrasions, iritis and iatrogenic damage to the retina and lens, with possible retinal detachment. The likelihood of these complications occurring increases as the number of intravitreal administrations performed on the same patient increases (Nguyen Q D, Rodrigues E B, Farah M E, Mieler W F, Do D V (eds): Retinal Pharmacotherapeutics. Dev Ophthalmol. Basel, Karger, 2016, vol 55, pp 63-70; Michael S. Ramos, et al., Ophthalmology Retina, Volume 5, Issue 7, 2021, pp. 625-632).

Because of this, intravitreal injection is not indicated in some pathological settings, such as glaucoma. In fact, the risk for post-injection IOP elevation is higher for patients with glaucoma or ocular hypertension. Good et al. demonstrated that patients with pre-existing glaucoma treated with intravitreal injections experience higher rates of elevated IOP when compared to patients without pre-existing glaucoma. It is possible that eyes with an already compromised aqueous humour outflow system may be more prone to developing elevated IOP in this setting (Good et al., Br J Ophthalmol 2011 August; 95 (8): 1111-4).

It is therefore felt the need to deliver therapeutically effective amounts of NGF to the eye using alternative routes of administration to the above-discussed routes, to ensure greater compliance with therapy by patients and to minimize the risks associated with administration.

### SUMMARY OF THE INVENTION

The present inventors have now surprisingly found that, when nerve growth factor (NGF) is administered intranasally, it reaches the retina and the optic nerve at therapeutically effective amounts. Thus, the intranasal administration of NGF can be used for the prevention or treatment of disorders of the retina and of the optic nerve.

Accordingly, a first object of the invention is nerve growth factor (NGF) for use in the prevention or treatment of a retinopathy or of an optic nerve disorder in a subject, wherein said nerve growth factor (NGF) is administered intranasally to the subject.

A further object of the invention is a mutein of nerve growth factor (NGF) for use in the prevention or treatment of a retinopathy or of an optic nerve disorder in a subject, wherein said mutein of nerve growth factor (NGF) is administered intranasally to the subject.

A further object of the invention is a pharmaceutical composition comprising nerve growth factor (NGF) and a pharmaceutically acceptable excipient or carrier, for use in the prevention or treatment of a retinopathy or of an optic nerve disorder in a subject, wherein said pharmaceutical composition is administered intranasally to the subject.

A further object of the invention is a pharmaceutical composition comprising a mutein of nerve growth factor (NGF) and a pharmaceutically acceptable excipient or carrier, for use in the prevention or treatment of a retinopathy or of an optic nerve disorder in a subject, wherein said pharmaceutical composition is administered intranasally to the subject.

A further object of the invention is the use of nerve growth factor (NGF) in the manufacture of a medicament for the prevention or treatment of a retinopathy or of an optic nerve disorder in a subject, wherein said nerve growth factor (NGF) is administered intranasally to the subject.

A further object of the invention is the use of a mutein of nerve growth factor (NGF) in the manufacture of a medicament for the prevention or treatment of a retinopathy or of an optic nerve disorder in a subject, wherein said mutein of nerve growth factor (NGF) is administered intranasally to the subject.

A further object of the invention is a method of preventing or treating a retinopathy or an optic nerve disorder in a subject, comprising intranasally administering to the subject a prophylactically or a therapeutically effective amount of nerve growth factor (NGF).

A further object of the invention is a method of preventing or treating a retinopathy or an optic nerve disorder in a subject, comprising intranasally administering to the subject a prophylactically or a therapeutically effective amount of a mutein of nerve growth factor (NGF).

### BRIEF DESCRIPTION OF THE FIGURES

FIG. 1 shows the rhNGF concentration found in the retinas and in the optic nerves of mice treated in accordance with Example 1 below.

FIG. 2 shows the graph of activated Caspase 3+ (% Area) in rat retina of CTRL (Healthy rats) or ONC (Optic Nerve Crush) experimental group with IN (intranasal) administration of Vehicle or rhNGF 0.3 mg/ml or rhNGF 2.14 mg/ml. CTRL=untreated retina, Vehicle=retina following Vehicle IN daily administration after ONC, rhNGF 0.3 mg/ml=retina following rhNGF 0.3 mg/ml IN daily administration after ONC, rhNGF 2.14 mg/ml=retina following rhNGF 2.14 mg/ml IN daily administration after ONC. Statistical significance is indicated with ***p<0.0001.

FIG. 3 shows the graph of RBMPS (RNA-binding protein with multiple splicing)+ (% Area) in rat retina of CTRL (Healthy rats) or ONC (Optic Nerve Crush) experimental group with IN (intranasal) administration of Vehicle or rhNGF 0.3 mg/ml or rhNGF 2.14 mg/ml. CTRL=untreated retina, Vehicle=retina following Vehicle IN daily administration after ONC, rhNGF 0.3 mg/ml=retina following rhNGF 0.3 mg/ml daily IN administration after ONC, rhNGF 2.14 mg/ml=retina following rhNGF 2.14 mg/ml IN daily administration after ONC. Statistical significance is indicated with **p<0.05.

FIG. 4 shows the graph of RBMPS (RNA-binding protein with multiple splicing)+ (% Area) in rat retina of CTRL (Healthy rats) or ONC (Optic Nerve Crush) experimental group with IN (intranasal) administration of Vehicle or rhNGF 0.075 mg/ml or 0.3 mg/ml with continuous or intermittent administration. CTRL=untreated retina, Vehicle=retina following Vehicle IN administration after ONC, rhNGF 0.075 mg/ml=retina following rhNGF 0.075 mg/ml IN continuous administration after ONC, rhNGF 0.3 l mg/ml=retina following rhNGF 0.3 mg/ml IN intermittent administration after ONC, rhNGF 0.3 C mg/ml=retina following rhNGF 0.3 mg/ml IN continuous administration after ONC. Statistical significance is indicated with *p<0.05, **p<0.01, ***p<0.001.

FIG. 5 shows the graph of Tuj1+ (% Area) in rat retina of CTRL (Healthy rats) or ONC (Optic Nerve Crush) experimental group with IN (intranasal) administration of Vehicle or rhNGF 0.075 mg/ml or 0.3 mg/ml with continuous or intermittent administration. CTRL=untreated retina, Vehicle=retina following Vehicle IN administration after ONC, rhNGF 0.075 mg/ml=retina following rhNGF 0.075 mg/ml IN continuous administration after ONC, rhNGF 0.3 l mg/ml=retina following rhNGF 0.3 mg/ml IN intermittent administration after ONC, rhNGF 0.3 C mg/ml=retina following rhNGF 0.3 mg/ml IN continuous administration after ONC. Statistical significance is indicated with *p<0.05, **p<0.01, ***p<0.001.

FIG. 6 shows the graph of Pearson R, indicating the degree of colocalization between the two RGC markers, RBPMS and Tuj1.

FIG. 7 shows the graph of RBPMS+ cells/mm² in rat retina of CTRL (Healthy rats) or ONC (Optic Nerve Crush) experimental group with IN (intranasal) administration of Vehicle or rhNGF 0.075 mg/ml or 0.3 mg/ml with continuous or intermittent administration. CTRL=untreated retina, Vehicle=retina following Vehicle IN administration after ONC, 0.075 mg/ml=retina following rhNGF 0.075 mg/ml IN continuous administration after ONC, 0.3 mg/ml INT=retina following rhNGF 0.3 mg/ml IN intermittent administration after ONC, 0.3 mg/ml CONT=retina following rhNGF 0.3 mg/ml IN continuous administration after ONC.

### DETAILED DESCRIPTION OF THE INVENTION

The present invention relates to nerve growth factor (NGF) for use in the prevention or treatment of a retinopathy or of an optic nerve disorder in a subject, wherein said nerve growth factor (NGF) is administered intranasally to the subject.

The term “prevention”, as used herein, refers to the inhibition of onset of the disorder or to the delay in the onset of the disorder.

Thus, according to a preferred embodiment, said nerve growth factor (NGF) is for use in the inhibition of onset or in the delay in the onset of a retinopathy or of an optic nerve disorder in a subject.

The term “treatment”, as used herein, refers to the amelioration or the inhibition of the progression or slowing of the progression of the disorder being treated.

Thus, according to a preferred embodiment, said nerve growth factor (NGF) is for use in the amelioration, the inhibition of the progression or the slowing of the progression of a retinopathy or of an optic nerve disorder in a subject.

Preferably, the term “amelioration of the disorder being treated”, as used herein, refers to the reduction of severity of the disorder being treated or of one or more of the symptoms associated thereof.

Thus, according to a preferred embodiment, said nerve growth factor (NGF) is for use in the reduction of the severity of a retinopathy or of an optic nerve disorder or of one of more of the symptoms associated thereof in a subject.

Preferably, the term “treatment” as used herein refers to the improvement of one or more of the following: visual function, visual acuity, structural changes of the optic nerve and/or retina.

Preferably, said retinopathy is selected from the group consisting of diabetic retinopathy, toxic retinopathy, diabetic macular edema, retinitis pigmentosa, retinal degeneration, retinal ischemia, phototoxic retinopathy, macular hole, epiretinal membrane, macular telangiectasia, retinopathy of prematurity, retinal gliosis, cystoid macular edema, retinal dystrophy, retinal vascular occlusion, retinal detachment, and age-related macular degeneration.

More preferably, said retinopathy is selected from the group consisting of diabetic retinopathy, retinitis pigmentosa and macular telangiectasia.

Preferably, said optic nerve disorder is selected from the group consisting of glaucoma, non-arteritic anterior ischemic optic neuropathy (NAION), arteritic anterior ischemic optic neuropathy (AAION), toxic optic neuropathy, posterior ischemic optic neuropathy, optic pathway glioma, preferably optic pathway glioma-induced visual loss, traumatic optic neuropathy, optic neuritis, Neuromyelitis Optica Spectrum Disorder (NMOSD), Leber hereditary optic neuropathy, radiation-induced optic neuropathy, amblyopia, inflammatory optic neuritis, infectious optic neuritis, non-infectious optic neuritis, and optic nerve atrophy.

Preferably, said glaucoma is selected from open-angle glaucoma, normal-tension glaucoma and angle-closure glaucoma.

Preferably, said inflammatory optic neuritis is multiple sclerosis (MS) associated optic neuritis.

More preferably, said optic nerve disorder is selected from non-arteritic anterior ischemic optic neuropathy (NAION), multiple sclerosis (MS) associated optic neuritis and glaucoma, said glaucoma being preferably selected from open-angle glaucoma, normal-tension glaucoma and angle-closure glaucoma.

A particularly preferred optic nerve disorder is non-arteritic anterior ischemic optic neuropathy (NAION).

Preferably, said subject is a human subject.

According to an embodiment, said nerve growth factor (NGF) is administered for the first time to the subject within 14 days of onset of the symptoms of said retinopathy or said optic nerve disorder.

According to an embodiment, said optic nerve disorder is selected from the group consisting of non-arteritic anterior ischemic optic neuropathy (NAION), posterior ischemic optic neuropathy and arteritic anterior ischemic optic neuropathy (AAION), and said nerve growth factor (NGF) is administered for the first time to the subject within 14 days of onset of the symptoms of said optic nerve disorder.

According to an embodiment, said subject is between 50 and 80 years old.

According to an embodiment, said subject has Best Corrected Visual Acuity (BCVA) score in the affected eye of ≥15 letters and ≤65 letters before treatment as measured by the ETDRS chart.

According to an embodiment, said nerve growth factor (NGF) is for use in improving the visual acuity of the subject, wherein said improving the visual acuity is preferably measured as improvement in Best Corrected Visual Acuity (BCVA).

Preferably, said improvement in Best Corrected Visual Acuity (BCVA) is defined as a >10 letter gain, more preferably ≥15 letter gain, wherein said improvement is measured as using the Early Treatment Diabetic Retinopathy Study (ETDRS) chart.

According to an embodiment, said nerve growth factor (NGF) is for use in the improvement of the visual function of the subject, wherein said visual function is measured as visual field mean sensitivity in decibels.

Preferably, said improvement of the visual function of the subject is defined as a gain of ≥7 decibels in mean sensitivity on visual field.

According to an embodiment, said nerve growth factor (NGF) is for use in the improvement of the structural changes of optic nerve head and/or retina.

Preferably, said improvement of the structural changes of optic nerve head and/or retina is measured by optical coherence tomography (OCT), for example by evaluating one or more of the following parameters: thickness of Retinal Nerve Fiber Layer (RNFL), Thickness of the Ganglion Cell Layer (GCL), Ganglion Cell Complex (GCC), or GCL-IPL. Preferably, said nerve growth factor (NGF) for use according to the present invention is administered to the subject by daily administration for at least one week.

According to a preferred embodiment, said nerve growth factor (NGF) for use according to the present invention is administered to the subject by intermittent administration for at least 3 cycles, wherein each cycle comprises, preferably consists of, one week of daily administration with nerve growth factor (NGF) followed by one, two or three weeks of wash-out.

Preferably, said nerve growth factor (NGF) for use according to the present invention is administered to the subject by intermittent administration for at least 3 cycles, wherein each cycle comprises, preferably consists of, one week of daily administration with nerve growth factor (NGF) followed by one week of wash-out.

Preferably, said nerve growth factor (NGF) for use according to the present invention is administered to the subject by intermittent administration for at least 4 cycles, wherein each cycle comprises, preferably consists of, one week of daily administration with nerve growth factor (NGF) followed by one week of wash-out.

Preferably, said nerve growth factor (NGF) for use according to the present invention is administered to the subject by intermittent administration for 4 cycles, wherein each cycle comprises, preferably consists of, one week of daily administration with nerve growth factor (NGF) followed by one week of wash-out.

The term “wash-out” as used herein refers to a period in which nerve growth factor (NGF) is not administered to the subject.

According to another preferred embodiment, said nerve growth factor (NGF) for use according to the present invention is administered to the subject by daily administration for at least one month.

Preferably, said daily administration comprises from one to three intranasal administrations of nerve growth factor (NGF) per day.

More preferably, said daily administration comprises, preferably consists of, three intranasal administrations of nerve growth factor (NGF) per day.

According to a preferred embodiment, said daily administration comprises, preferably consists of, one intranasal administration of nerve growth factor (NGF) per day.

Preferably, the amount of nerve growth factor (NGF) per each intranasal administration is between 5 μg and 720 μg, more preferably between 10 μg and 400 μg, even more preferably between 15 μg and 200 μg, most preferably about 60 μg.

According to a preferred embodiment, the amount of nerve growth factor (NGF) per daily administration is between 15 μg and 2100 μg, more preferably between 30 μg and 1200 μg, even more preferably between 100 μg and 300 μg, most preferably about 180 μg, divided equally in each nostril.

According to a preferred embodiment a liquid (e.g., aqueous) pharmaceutical composition containing the nerve growth factor (NGF) and at least one pharmaceutically acceptable excipient is intranasally administered to the patient. Preferably, the pharmaceutical composition consists essentially of nerve growth factor (NGF). Preferably nerve growth factor (NGF) is the only active ingredient in the pharmaceutical composition. According to a preferred embodiment, the pharmaceutical composition does not contain one or more of brain derived neurotrophic growth factor (BDNF), transforming growth factor (TGF, e.g., TGF-alpha, TGF-beta), epidermal growth factor (EGF), platelet derived growth factor (PDGF), and an interleukin (e.g., IL-1, IL-4, IL-6, IL-10 and IL-13).

The effective amount of said nerve growth factor (NGF) used in each administration, the duration of the treatment therapy, and the number of administrations per day are selected by the skilled person based on the characteristics and on the disorder of the subject to be treated, the severity of the symptoms and based on assessment tests carried out during the treatment.

Preferably, said nerve growth factor (NGF) for use according to the present invention is human nerve growth factor (hNGF).

Preferably, said human nerve growth factor (hNGF) has an aminoacid sequence consisting of SEQ. ID NO. 1 below

SEQ. ID NO. 1:
SSSHPIFHRGEFSVCDSVSVWVGDKTTATDIKGKEVMVLGEVNINNSVF
KQYFFETKCRDPNPVDSGCRGIDSKHWNSYCTTTHTFVKALTMDGKQAA
WRFIRIDTACVCVLSRKAVR;

or
SEQ ID NO. 2 below:

SEQ ID NO. 2:
SSSHPIFHRGEFSVCDSVSVWVGDKTTATDIKGKEVMVLGEVNINNSVF
KQYFFETKCRDPNPVDSGCRGIDSKHWNSYCTTTHTFVKALTMDGKQAA
WRFIRIDTACVCVLSRKAVRRA

In certain embodiments nerve growth factor comprises the amino acid sequence of SEQ ID NO: 1.

More preferably, said human nerve growth factor (NGF) has the aminoacid sequence of SEQ. ID NO. 1.

The human nerve growth factor having the aminoacid sequence of SEQ. ID. NO. 1 is advantageous because it is associated with an improved pharmacological profile in the context of the diseases of the invention. Indeed, as disclosed in WO2022167607A1, the human nerve growth factor having the aminoacid sequence of SEQ. ID. NO. 1 predominantly activates TrKA-mediated pathways and inhibits apoptotic pathways mediated by p75^{NTR}, and thus is particularly useful in the treatment of pathologies where the effects of NGF on proliferation and survival are desired and where the proapoptotic effect of p75^{NTR} is detrimental.

Preferably, the nerve growth factor (NGF) for use according to the present invention is produced by recombinant DNA technology, preferably it is a human recombinant nerve growth factor (rhNGF).

Methods of producing rhNGF are known to the person skilled in the art, for example those described in WO0022119A1 and WO2013092776A1.

Preferably, the nerve growth factor (NGF) for use according to the present invention has a purity higher than 70%, more preferably higher than 80%, higher than 90%, higher than 95%, higher than 98% or higher than 99%. The purity of nerve growth factor (NGF) may be determined by conventional means known to those skilled in the art, preferably by HPLC analysis.

Muteins of nerve growth factor (NGF) have been described in the art.

Accordingly, a further object of the present invention relates to a mutein of nerve growth factor for use in the prevention or treatment of a retinopathy or of an optic nerve disorder in a subject, wherein said mutein of nerve growth factor (NGF) is administered intranasally to the subject.

Preferably, said retinopathy is as described above.

Preferably, said optic nerve disorder is as described above.

A particularly preferred optic nerve disorder is non-arteritic anterior ischemic optic neuropathy (NAION).

Preferably, said “prevention” and “treatment” are as defined above.

Preferably, said subject is as described above.

Preferably, said mutein of nerve growth factor (NGF) is administered to the subject for the first time to the subject within 14 days of onset of the symptoms of said retinopathy or said optic nerve disorder.

According to an embodiment, said optic nerve disorder is selected from the group consisting of non-arteritic anterior ischemic optic neuropathy (NAION), posterior ischemic optic neuropathy and arteritic anterior ischemic optic neuropathy (AAION), and said mutein of nerve growth factor (NGF) is administered for the first time to the subject within 14 days of onset of the symptoms of said optic nerve disorder.

Preferably, said mutein of nerve growth factor (NGF) is for use in one or more of the following: improving the visual acuity of the subject, improving the visual function of the subject, improving the structural changes of optic nerve head and/or retina.

Preferably, said visual acuity, said visual function and said structural changes of optic nerve head and/or retina are measured as described above, and said improvement of each parameter is defined as disclosed above.

Preferably, the mutein of nerve growth factor (NGF) for use according to the present invention is administered to the subject as disclosed above in relation to nerve growth factor (NGF).

Preferably, the amount of mutein of nerve growth factor (NGF) for use according to the present invention per daily administration is as disclosed above in relation to nerve growth factor (NGF).

Preferably, said mutein of nerve growth factor (NGF) for use according to the present invention is a biologically active mutein of nerve growth factor (NGF), meaning a nerve growth factor (NGF) protein having an amino acid sequence with one or more amino acid mutations, preferably substitutions, such that the therapeutic activity of wild type nerve growth factor (NGF) is maintained.

Preferably, said mutein of nerve growth factor (NGF) for use according to the present invention maintains the biological activity of nerve growth factor (NGF).

Preferably, said mutein of nerve growth factor (NGF) for use according to the present invention maintains the biological activity of nerve growth factor (NGF), and has one or more of the following properties compared to nerve growth factor (NGF): reduced side effects, improved pharmacokinetic, selective binding, enhanced potency.

Preferably, said mutein of nerve growth factor (NGF) for use according to the present invention is a mutein of wild type human nerve growth factor (NGF), as described above. According to an embodiment, said mutein of nerve growth factor (NGF) for use according to the present invention has an aminoacid sequence with preferably more than 90%, more preferably more than 95%, even more preferably more than 98% identity with SEQ ID NO. 1 or SEQ ID NO. 2.

According to an embodiment, said mutein of nerve growth factor (NGF) for use according to the present invention has an aminoacid sequence differing from SEQ ID NO. 1 or SEQ ID NO. 2 for the identity of between 1 and 5 aminoacids.

According to an embodiment, said mutein of nerve growth factor (NGF) for use according to the present invention has an aminoacid sequence consisting of SEQ ID NO. 1 or SEQ ID NO. 2 wherein the aminoacid at position 61 is substituted by a different aminoacid.

Preferably, said mutein of nerve growth factor (NGF) for use according to the present invention contains a serine at a position corresponding to position 61 of SEQ ID NO. 1 or SEQ ID NO. 2.

According to an embodiment, said mutein of nerve growth factor (NGF) for use according to the present invention has an aminoacid sequence consisting of SEQ ID NO. 1 or SEQ ID NO. 2 wherein at least one aminoacid at a position corresponding to positions 95-101 is substituted by a different aminoacid.

Preferably, said mutein of nerve growth factor (NGF) for use according to the present invention has an aminoacid sequence consisting of SEQ ID NO. 1 or SEQ ID NO. 2 wherein at least one aminoacid at a position corresponding to positions 95-101 is substituted by a different aminoacid, wherein said mutein contains a glutamate at a position corresponding to position 100 of SEQ ID NO. 1 or SEQ ID NO. 2.

According to an embodiment, said mutein of nerve growth factor (NGF) for use according to the present invention has an aminoacid sequence consisting of SEQ ID NO. 1 or SEQ ID NO. 2 wherein the aminoacid at positions 100 is substituted by a different aminoacid.

Preferably, said mutein of nerve growth factor (NGF) for use according to the present invention contains a glutamate at a position corresponding to position 100 of SEQ ID NO. 1 or SEQ ID NO. 2.

According to an embodiment, said mutein of nerve growth factor (NGF) for use according to the present invention has an aminoacid sequence consisting of SEQ ID NO. 1 or SEQ ID NO. 2 wherein the aminoacid at a position corresponding to position 61 of SEQ ID NO. 1 or SEQ ID NO. 2 is serine and the aminoacid at a position corresponding to position 100 of SEQ ID NO. 1 or SEQ ID NO. 2 is glutamate.

Particularly preferred muteins for use according to the present invention have the amino acid sequences consisting of SEQ ID NO:3-6 below

SEQ ID NO: 3:
SSSHPIFHRGEFSVCDSVSVWVGDKTTATDIKGKEVMVLGEVNINNSVF
KQYFFETKCRDPNPVDSGCRGIDSKHWNSYCTTTHTFVKALTMDGKQAA
WEFIRIDTACVCVLSRKAVR
SEQ ID NO: 4:
SSSHPIFHRGEFSVCDSVSVWVGDKTTATDIKGKEVMVLGEVNINNSVF
KQYFFETKCRDPNPVDSGCRGIDSKHWNSYCTTTHTFVKALTMDGKQAA
WEFIRIDTACVCVLSRKAVRRA
SEQ ID NO: 5:
SSSHPIFHRGEFSVCDSVSVWVGDKTTATDIKGKEVMVLGEVNINNSVF
KQYFFETKCRDSNPVDSGCRGIDSKHWNSYCTTTHTFVKALTMDGKQAA
WEFIRIDTACVCVLSRKAVR
SEQ ID NO: 6:
SSSHPIFHRGEFSVCDSVSVWVGDKTTATDIKGKEVMVLGEVNINNSVF
KQYFFETKCRDSNPVDSGCRGIDSKHWNSYCTTTHTFVKALTMDGKQAA
WEFIRIDTACVCVLSRKAVRRA

Preferred muteins comprise the amino acid sequence of SEQ ID NO: 3 or 5.

According to an embodiment, said mutein of human nerve growth factor (NGF) for use according to the present invention is produced by recombinant DNA technology. Methods of producing muteins of rhNGF for use according to the invention by recombinant DNA technology are known to the person skilled in the art.

A further object of the present invention relates to a pharmaceutical composition comprising nerve growth factor (NGF) and at least one pharmaceutically acceptable excipient, for use in the prevention or treatment of a retinopathy or of an optic nerve disorder in a subject, wherein said pharmaceutical composition is administered intranasally to the subject.

Preferably, said nerve growth factor (NGF) is as described above.

Preferably, said retinopathy is selected from the group consisting of diabetic retinopathy, toxic retinopathy, diabetic macular edema, retinitis pigmentosa, retinal degeneration, retinal ischemia, phototoxic retinopathy, macular hole, epiretinal membrane, macular telangiectasia, retinopathy of prematurity, retinal gliosis, cystoid macular edema, retinal dystrophy, retinal vascular occlusion, retinal detachment, and age-related macular degeneration.

More preferably, said retinopathy is selected from the group consisting of diabetic retinopathy, retinitis pigmentosa and macular telangiectasia.

Preferably, said optic nerve disorder is selected from the group consisting of glaucoma, non-arteritic anterior ischemic optic neuropathy (NAION), arteritic anterior ischemic optic neuropathy (AAION), toxic optic neuropathy, posterior ischemic optic neuropathy, optic pathway glioma, preferably optic pathway glioma-induced visual loss, traumatic optic neuropathy, optic neuritis, Neuromyelitis Optica Spectrum Disorder (NMOSD), Leber hereditary optic neuropathy, radiation-induced optic neuropathy, amblyopia, inflammatory optic neuritis, infectious optic neuritis, non-infectious optic neuritis, and optic nerve atrophy.

Preferably, said glaucoma is selected from open-angle glaucoma, normal-tension glaucoma and angle-closure glaucoma.

Preferably, said inflammatory optic neuritis is multiple sclerosis (MS) associated optic neuritis.

More preferably, said optic nerve disorder is selected from non-arteritic anterior ischemic optic neuropathy (NAION), multiple sclerosis (MS) associated optic neuritis and glaucoma, said glaucoma being preferably selected from open-angle glaucoma, normal-tension glaucoma and angle-closure glaucoma.

According to a preferred embodiment, the pharmaceutical composition for use according to the present invention consists essentially of nerve growth factor (NGF).

Preferably, nerve growth factor (NGF) is the only active ingredient in the pharmaceutical composition for use according to the present invention.

According to a preferred embodiment, the pharmaceutical composition for use according to the present invention does not contain one or more of brain derived neurotrophic growth factor (BDNF), transforming growth factor (TGF, e.g., TGF-alpha, TGF-beta), epidermal growth factor (EGF), platelet derived growth factor (PDGF), and an interleukin (e.g., IL-1, IL-4, IL-6, IL-10 and IL-13).

Preferably, the pharmaceutical composition for use according to the invention is a liquid composition suitable for intranasal administration (liquid intranasal composition).

Preferably, the pharmaceutical composition for use according to the present invention comprises nerve growth factor (NGF) and at least one pharmaceutically acceptable excipient suitable for intranasal administration.

Preferably, said at least one pharmaceutically acceptable excipient suitable for intranasal administration is selected from solvents, thickening agents, mucoadhesive agents, buffers, antioxidants, preservatives, and penetration enhancers.

Preferably, said pharmaceutical composition for use according to the present invention is administered for the first time to the subject within 14 days of onset of the symptoms of said retinopathy or said optic nerve disorder.

Preferably, said optic nerve disorder is selected from the group consisting of non-arteritic anterior ischemic optic neuropathy (NAION), posterior ischemic optic neuropathy and arteritic anterior ischemic optic neuropathy (AAION), and said pharmaceutical composition for use according to the present invention is administered for the first time to the subject within 14 days of onset of the symptoms of said optic nerve disorder.

Preferably, the pharmaceutical composition for use according to the present invention is administered to the subject by daily administration for at least one week.

According to a preferred embodiment, the pharmaceutical composition for use according to the invention is administered to the subject by intermittent administration for at least 3 cycles, wherein each cycle comprises, preferably consists of, one week of daily administration with the pharmaceutical composition followed by one, two or three weeks of wash-out.

According to a preferred embodiment, the pharmaceutical composition for use according to the invention is administered to the subject by intermittent administration for at least 3 cycles, wherein each cycle comprises, preferably consists of, one week of daily administration with the pharmaceutical composition followed by one week of wash-out.

According to a preferred embodiment, the pharmaceutical composition for use according to the invention is administered to the subject by intermittent administration for at least 4 cycles, wherein each cycle comprises, preferably consists of, one week of daily administration with the pharmaceutical composition followed by one week of wash-out.

According to a preferred embodiment, the pharmaceutical composition for use according to the invention is administered to the subject by intermittent administration for 4 cycles, wherein each cycle comprises, preferably consists of, one week of daily administration with the pharmaceutical composition followed by one week of wash-out.

The term “wash-out” as used herein refers to a period in which the pharmaceutical composition is not administered to the subject.

According to another preferred embodiment, the pharmaceutical composition for use according to the invention is administered to the subject by daily administration for at least one month.

Preferably, said daily administration comprises from one to three intranasal administrations of the pharmaceutical composition per day.

More preferably, said daily administration comprises, preferably consists of, three intranasal administrations of the pharmaceutical composition per day.

According to another preferred embodiment, said daily administration comprises, preferably consists of, one intranasal administration of the pharmaceutical composition per day.

Based on the volume to be administered, the administration may be split in two separate doses to be administered not more than 20 minutes, preferably between 5 and 15 minutes, one from the other.

Preferably, the concentration of said nerve growth factor (NGF) in said liquid intranasal composition is between 20 μg/ml and 180 μg/ml, more preferably about 60 μg/ml.

According to a preferred embodiment, the concentration of said nerve growth factor (NGF) in said liquid intranasal composition is between 80 μg/ml and 240 μg/ml, more preferably about 180 μg/ml.

The pharmaceutical composition for use according to the invention may be suitably formulated using appropriate methods known in the art or by the method disclosed in Remington's Pharmaceutical Science (recent edition), Mack Publishing Company, Easton Pa.

A further object of the present invention relates to a pharmaceutical composition comprising a mutein of nerve growth factor (NGF) and at least one pharmaceutically acceptable excipient, for use in the prevention or treatment of a retinopathy or of an optic nerve disorder in a subject, wherein said pharmaceutical composition is administered intranasally to the subject.

Preferably, said mutein of nerve growth factor (NGF) is as described above.

Preferably, said retinopathy and said optic nerve disorder are as described above.

Preferably, said pharmaceutical composition is as described above in relation to the pharmaceutical composition comprising nerve growth factor (NGF).

Preferably, the administration of the pharmaceutical composition is as described above in relation to the pharmaceutical composition comprising nerve growth factor (NGF).

Preferably, the amount concentration of said mutein of nerve growth factor (NGF) in the pharmaceutical composition is as disclosed above in relation to the pharmaceutical composition comprising nerve growth factor (NGF).

In a further aspect, the present invention relates to the use of nerve growth factor (NGF) in the manufacture of a medicament for the prevention or treatment of a retinopathy or of an optic nerve disorder in a subject, wherein said nerve growth factor (NGF) or mutein thereof is administered intranasally to the subject.

In a further aspect, the present invention relates to the use of a mutein of nerve growth factor (NGF) in the manufacture of a medicament for the prevention or treatment of a retinopathy or of an optic nerve disorder in a subject, wherein said mutein of nerve growth factor (NGF) is administered intranasally to the subject.

In a further aspect, the present invention relates to a method for the prevention or treatment of a retinopathy or of an optic nerve disorder in a subject in need thereof, wherein the method comprises intranasally administering nerve growth factor (NGF) to the subject in a prophylactically or therapeutically effective amount.

In a further aspect, the present invention relates to a method for the prevention or treatment of a retinopathy or of an optic nerve disorder in a subject in need thereof, wherein the method comprises intranasally administering a mutein of nerve growth factor (NGF) to the subject in a prophylactically or therapeutically effective amount.

A “prophylactically effective amount” according to the present invention means an amount sufficient to achieve prevention of the disease.

A “therapeutically effective amount” according to the present invention means an amount sufficient to achieve treatment of the disease.

Preferably, said nerve growth factor (NGF) or mutein thereof is in the form of a pharmaceutical composition, as above described.

The invention will be further described in the following examples, which do not limit the scope of the invention described in the claims.

### EXPERIMENTAL SECTION

The present inventors have carried out experiments by administering NGF intranasally to different animal models. The concentration of NGF has been determined in the retina and in the optic nerve of the animals after intranasal administration to evaluate the feasibility of this route of administration to deliver NGF to these ocular structures in therapeutically effective amounts.

### Example 1

A biodistribution study of rhNGF in the optic nerve and in the retina at 2 hours, 12 hours, 24 hours and 48 hours post-intranasal administration in mice was carried out as follows.

### Animals

C57BL/6 male mice were used. Mice were housed in macrolon cages with filter hoods, in a room where the air was continuously filtered, thereby avoiding contamination. Animals arrived on site four days before rhNGF administration to allow optimal acclimation. During experiments, paired animals were caged at a constant temperature with a day/night cycle of 12/12 hours. Animals received water (control tap water) and nutrition ad libitum. Animal protocol was approved by the Animal Studies Committee of Languedoc Roussillon. This protocol and the procedures used comply with French legislation, which implements the European Directives (Reference Number: D3417223, APAFIS #23920-2020020320279696 v3). Two experimental sessions were carried out to cover the entire time course. To merge the obtained data, vehicle-treated animals of each experimental session, having a comparable ELISA response, were mediated. Mice were 10 weeks old and were randomly divided into 6 groups:
1) vehicle, which received only saline buffer; sampling at 2 h (n=3)
2) rhNGF-treated; sampling at 2 h (n=4)
3) vehicle, which received only saline buffer; sampling at 12 h (n=3)
4) rhNGF-treated; sampling at 12 h (n=4)
5) rhNGF-treated; sampling at 24 h (n=4)
6) rhNGF-treated; sampling at 48 h (n=4)

### Intranasal Compound Administration

rhNGF 2 mg/ml was administered by intranasal route at 10 μl/animal. Using a dominant hand, the micropipette was leaded with 10 μl of compound or vehicle. The tip of the filled pipette was placed near the mouse's left nostril at 45-degree angle. The droplet was placed close enough to the mouse's nostril so that the mouse can inhale the droplet. Immediately after the mouse inhaled this small drop, the rest of the compound in the pipette tip was ejected to form another small droplet for the mouse to inhale through the same nostril about 2-3 sec later. After administration, the mouse was held in this position for 15 sec.

### Organ Sampling

The temporal bone was opened, and the left optic nerves of all animals were sampled. Each sample was rapidly frozen using liquid nitrogen and stored at −80° C. Then, the connective tissue in the orbital cavity surrounding the eye and any remaining tissue were cut to extract the intact eyeball. The retina was sampled using a scalpel and quickly frozen in liquid nitrogen and stored at −80° C. Frozen tissues were cut into small parts, solubilized in 15 μl of sterile PBS completed with protease inhibitors (Fisher Scientific, France) and sonicated 3 times during 10 seconds on ice (Microson ultrasonic cell disruptorXL, Microsonic). Then, homogenates were centrifuged for 30 minutes at 10,000 rpm at 4° C., and 10 μl of supernatant stored at −20° C. before rhNGF ELISA analysis.

rhNGF Analysis by ELISA

10 μl of diluted samples at 1/10 for the optic nerve and at 1/10 or ⅕ for retina were analyzed by ELISA method (Novus Biological, Ref. NBP2-62776). Standard references were diluted at 1000, 500, 250, 125, 62.5, 31.25, 15.63 and 0 μg/mL to perform the standard curve. The Standard working solution of different concentrations was added to the first two columns: each concentration of the solution was added into two wells side by side and the samples to other wells. The plate was covered with sealer provided in the kit and incubated for 90 min at 37° C. Then, the liquid of each well was removed and immediately 100 μL of Biotinylated Detection Ab working solution was added to each well. The plate was again covered with sealer provided in the kit and incubated for 1 hour at 37° C. After incubation, the solution from each well was aspirated and 350 μL of wash buffer was added and aspirated to each well. This wash step was repeated 3 times. 100 μL of HRP-conjugated working solution was added to each well. The plate was again covered with sealer provided in the kit and incubated for 30 min at 37° C. After washing, 90 μL of Substrate Reagent was added to each well and incubated for 15 min at 37° C. Finally, 50 μL of Stop Solution was added to each well and the optical density of each well was immediately determined using a microplate reader set to 450 nm.

### Results

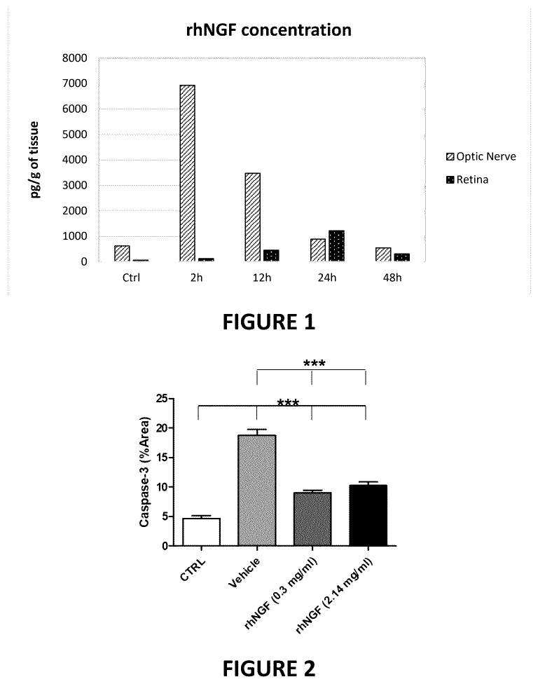

The results are summarized in Table 1 below, which reports the mean rhNGF concentrations (pg/g of tissue) detected in the optic nerve and in the retina at the different time points analyzed, and in FIG. 1.

TABLE 1

| | | Time after treatment | | | |
|---|---|---|---|---|---|
| Tissue | Group | 2 hours | 12 hours | 24 hours | 48 hours |
| Optic Nerve | Control | 615 | 615 | 615 | 615 |
| | (mean 2 | | | | |
| | and 12 h) | | | | |
| | Treated | 6923 | 3473 | 878 | 540 |
| Retina | Control | 55 | 55 | 55 | 55 |
| | (mean 2 | | | | |
| | and 12 h) | | | | |
| | Treated | 120 | 450 | 1210 | 305 |

A relevant increase of rhNGF concentration was observed in optic nerve of rhNGF treated group compared to the control group. 2 hours after single rhNGF administration by intranasal route, rhNGF was detected in optic nerve at a mean of 6923 μg/g of tissue. Progressive decrease of rhNGF concentration was observed at 12 hours, 24 hours and 48 hours post-treatment with a concentration of 3473 μg/g, 878 μg/g and 540 μg/g respectively. Taken together, these results demonstrate that rhNGF reaches the optic nerve when administered by intranasal route, with a peak concentration at 2 hours post treatment.

A slight increase of rhNGF concentration was observed in the retina of rhNGF-treated animals 2 hours post-administration compared to control animals. A relevant increase of rhNGF concentration was observed in the retina at 12 hours and 24 hours post-treatment in the rhNGF-treated group compared to the vehicle-treated group. At 12 h post-administration, the compound was detected at 450 μg/g of tissue and at 24 h post-administration the compound was detected at 1210 μg/g. The progressive increase of rhNGF concentration observed from 12 h to 24 h post-treatment reached a peak at 24 h post-treatment. Then, decrease of rhNGF concentration was observed at 48 h post-treatment, still remaining higher than the basal levels.

### Example 2

A biodistribution study of rhNGF in the optic nerve and in the retina in New Zealand White Rabbits was carried out as follows.

New Zealand White rabbits (n=10) were treated either with control (physiological saline solution) or with rhNGF. The animals were divided into the following groups:
Group 1 (control): animals treated with physiological saline solution, n=2;
Group 2 (treatment): animals treated with rhNGF 0.6 mg/mL, n=4;
Group 3 (treatment): animals treated with rhNGF 1.2 mg/mL, n=4.

The administration was carried out intranasally (0.5 mL/nostril) once a day, from day 1 to day 7. The animals were sacrificed 6±1 hours after the last administration on day 7.

Optic nerve and retina were homogenized before analysis: each optic nerve and retina samples was thawed in ice and 500 μL of Lysis buffer were added. Then, each sample was homogenized in ice by ultraturrax (3 cycles 30 sec On/30 sec Off at about 21000 g/min) and centrifuged for 15 minutes at 20000×g, 4° C. Supernatants were frozen at −70°±10° C. until analysis.

The results are shown in Table 2 below, which reports the rhNGF mean level (ng of rhNGF/g of tissue) detected in the optic nerve and in the retina of the analyzed animals.

TABLE 2

| | | | rhNGF level | rhNGF mean |
|---|---|---|---|---|
| | | | (ng of | level (ng of |
| | | | protein/g of | protein/g of |
| Tissue | Group | Animal n. | tissue) | tissue) |
| Optic | 1 | 12 right | 0.0 | 1.9 |
| Nerve | (Control) | 12 left | 1.4 | |
| | | 22 right | 3.7 | |
| | | 22 left | 2.6 | |
| | 2 | 15 right | 4.1 | 3.6 |
| | (rhNGF 0.6 | 15 left | 2.1 | |
| | mg/mL) | 16 right | 1.8 | |
| | | 16 left | 0.0 | |
| | | 25 right | 4.1 | |
| | | 25 left | 6.0 | |
| | | 26 right | 2.9 | |
| | | 26 left | 7.5 | |
| | 3 | 19 right | 9.5 | 6.9 |
| | (rhNGF 1.2 | 19 left | 8.3 | |
| | mg/mL) | 20 right | 3.8 | |
| | | 20 left | 2.9 | |
| | | 29 right | 0.0 | |
| | | 29 left | 5.7 | |
| | | 30 right | 16.3 | |
| | | 30 left | 9.1 | |
| Retina | 1 | 12 right | 3.1 | 3.8 |
| | (Control) | 12 left | 4.0 | |
| | | 22 right | 1.4 | |
| | | 22 left | 6.6 | |
| | 2 | 15 right | 6.2 | 4.9 |
| | (rhNGF 0.6 | 15 left | 3.9 | |
| | mg/mL) | 16 right | 3.1 | |
| | | 16 left | 1.8 | |
| | | 25 right | 1.9 | |
| | | 25 left | 11.9 | |
| | | 26 right | 3.0 | |
| | | 26 left | 7.1 | |
| | 3 | 19 right | 2.3 | 4.4 |
| | (rhNGF 1.2 | 19 left | 1.7 | |
| | mg/mL) | 20 right | 3.2 | |
| | | 20 left | 4.5 | |
| | | 29 right | 4.6 | |
| | | 29 left | 2.2 | |
| | | 30 right | 6.5 | |
| | | 30 left | 9.9 | |

A relevant increase of rhNGF concentration was observed in optic nerve of rhNGF treated groups compared to the control group. The increase of rhNGF concentration in the optic nerve was dose-dependent: an almost double concentration of rhNGF was found in the optic nerve of group 3, which was treated with rhNGF 1.2 mg/mL, compared to group 2, which was treated with rhNGF 0.6 mg/ml (3.6 ng of rhNGF/g of tissue for group 2 vs. 6.9 ng of rhNGF/g of tissue for group 3).

A trend of increase of rhNGF concentration was found in the retina of rhNGF treated groups compared to the control group. This increase agrees with the data discussed in Example 1, where at the time immediately following administration there is only a slight trend of increase of rhNGF concentration in the retina, which then becomes relevant at longer times (12 h and 24 h).

### Example 3

A biodistribution study of rhNGF in the optic nerve and in the retina in New Zealand White Rabbits was carried out as follows.

New Zealand White rabbits (n=11) were treated either with control (physiological saline solution) or rhNGF. The animals were divided into the following groups:
Group 1 (control): animals treated with physiological saline solution, n=2;
Group 2 (treatment): animals treated with rhNGF 1.2 mg/mL, n=6;
Group 3 (treatment): animal treated with rhNGF 0.320 mg/mL, n=3.

A single administration was carried out intranasally (0.5 mL/nostril) at day 1. The animals were sacrificed 18±1 hours after the administration.

Optic nerve and retina samples were homogenized before analysis: each optic nerve and retina sample was thawed in ice and 500 μL of Lysis buffer were added. Then, each sample was homogenized in ice by ultraturrax (3 cycles 30 sec On/30 sec Off at about 21000 g/min) and centrifuged for 15 minutes at 20000×g, 4° C. Supernatants were frozen at −70°±10° C. until analysis.

The results are shown in Table 3 below, which reports the rhNGF mean levels detected in the optic nerve and in the retina of the analyzed animals.

TABLE 3

| | | | rhNGF | rhNGF mean |
|---|---|---|---|---|
| | | | level (ng of | level (ng |
| | | Animal | protein/ | of protein/ |
| Tissue | Group | n. | g of tissue) | g of tissue) |
| Optic | 1 | 1 right | 24.2 | 31.3 |
| Nerve | Control | 1 left | 55.1 | |
| | | 2 right | 10.5 | |
| | | 2 left | 35.2 | |
| | 2 | 3 right | 35.9 | 39.5 |
| | rhNGF 1.2 | 3 left | 10.9 | |
| | mg/mL | 4 right | 52.3 | |
| | | 4 left | 33.2 | |
| | | 5 right | 48.0 | |
| | | 5 left | 71.5 | |
| | | 6 right | 46.9 | |
| | | 6 left | 30.1 | |
| | | 7 right | 50.0 | |
| | | 7 left | 26.3 | |
| | | 8 right | 28.4 | |
| | | 8 left | 40.6 | |
| | 3 | 9 right | 35.7 | 32.7 |
| | rhNGF 0.320 | 9 left | 25.8 | |
| | mg/mL | 10 right | 32.9 | |
| | | 10 left | 30.8 | |
| | | 11 right | 30.1 | |
| | | 11 left | 41.0 | |
| Retina | 1 | 1 right | 21.0 | 15.5 |
| | Control | 1 left | 20.6 | |
| | | 2 right | 8.3 | |
| | | 2 left | 12.0 | |
| | 2 | 3 right | 8.2 | 17.0 |
| | rhNGF 1.2 | 3 left | 7.1 | |
| | mg/mL | 4 right | 12.7 | |
| | | 4 left | 18.6 | |
| | | 5 right | 16.1 | |
| | | 5 left | 14.9 | |
| | | 6 right | 14.8 | |
| | | 6 left | 12.4 | |
| | | 7 right | 19.8 | |
| | | 7 left | 42.6 | |
| | | 8 right | 28.2 | |
| | | 8 left | 8.9 | |
| | 3 | 9 right | 22.8 | 29.1 |
| | rhNGF 0.320 | 9 left | 14.1 | |
| | mg/ml | 10 right | 52.5 | |
| | | 10 left | 17.4 | |
| | | 11 right | 41.1 | |
| | | 11 left | 26.5 | |

Although some groups showed a trend of increase of rhNGF levels in the optic nerve of the treated animals compared to control (group 2), this increase was of limited entity. Since the evaluation was carried out 18 hours after administration, these data are in line with the results obtained in Example 1, where an increase in optic nerve protein concentration is observed immediately after administration (peak at 2 hours) and then a steady decrease is observed at subsequent time points. In light of the slight increase found in treated group 2 compared to control, it is reasonable to assume that the concentration of the protein in the optic nerve increased in the first few hours after administration and then went through a decrease, which has not yet been completely exhausted for group 2, where a slightly higher level of the protein is observed.

An increase of rhNGF levels in the retina was observed particularly in group 3, where the level of rhNGF detected almost doubled the levels detected in the control group. This is surprising, considering that group 3 was treated with a significantly lower rhNGF concentration than the other groups.

### Example 4

A preliminary biodistribution study of rhNGF in the optic nerve and in the retina in adult dogs was carried out as follows.

Three adult (5 to 7 months) Beagle male dogs (animal supplier: Marshall Bioresources) were used in the study.

One dog (control) was untreated.

The other two dogs (treatment 1 and treatment 2) were treated with rhNGF at the concentration of 1.2 mg/mL.

The administration of the liquid formulation was carried out intranasally (0.4 mL/nostril) via MAD (Mucosal Atomization Device).

Two administrations in each nostril were given 1 hour apart, and the dogs were sacrificed 30 minutes after the second administration. The total amount of rhNGF administered was 1.92 mg.

Optic nerve and retina were homogenized before analysis: each optic nerve and retina sample were thawed in ice and 500 μL of Lysis buffer were added. Then, each sample was homogenized in ice by ultraturrax (3 cycles 30 sec On/30 sec Off at about 21000 g/min) and centrifuged for 15 minutes at 20000×g, 4° C. Supernatants were frozen at −70°±10° C. until analysis.

The results are shown in Table 4 below, which reports the rhNGF mean levels detected in the optic nerve and in the retina of the analyzed animals.

TABLE 4

| | | | rhNGF level | Mean rhNGF |
|---|---|---|---|---|
| | | | (pg of | level (pg of |
| | | | protein/g of | protein/g of |
| Tissue | Group | Animal | tissue) | tissue) |
| Retina | Control | right | 266.4 | 227.7 |
| | | left | 188.9 | |
| | rhNGF 1.2 | 1 right | 268.2 | 332.8 |
| | mg/mL | 1 left | 273.0 | |
| | | 2 right | 436.0 | |
| | | 2 left | 353.8 | |
| Optic Nerve | Control | right | 118.3 | 148.0 |
| | | left | 177.7 | |
| | rhNGF 1.2 | 1 right | 181.4 | 218.2 |
| | mg/mL | 1 left | 285.1 | |
| | | 2 right | 188.6 | |
| | | 2 left | 217.8 | |

As can be seen, a slight increase of rhNGF level was found in the retinas and in the optic nerves. Even if the study was performed with a limited number of animals, it indicates that the rhNGF intranasally delivered by means of MAD device is absorbed and distributes in the back of the eye tissues.

### Example 5

A study was carried out in a rat model of Optic Nerve Crush (ONC) (Mesentier-Louro et al., Mol Neurobiol 56, 1056-1069 (2019)) to evaluate the neuroprotection of rhNGF intranasal treatment.

The ONC model is widely used in vision research to study various pathologies related to optic nerve injury and neurodegeneration. It replicates key pathological features common to various retinopathies and optic neuropathies, including RGC loss, axon degeneration, and glial activation. Following ONC, RGC degeneration closely mimics the progressive neuronal degeneration observed in diseases such as glaucoma and traumatic optic neuropathy (Tang Z, et al. J Vis Exp. 2011; Cammalleri, M., et al., Optic nerve crush as a model of retinal ganglion cell degeneration. 2022, Annals of Eye Science). The model also induces robust glial responses, and activation of pro-apoptotic pathways such as p38, JNK, and caspase-3, reflecting the neuroinflammatory and neurodegenerative mechanisms seen across diverse optic nerve pathologies. These features make ONC a robust and reproducible model to investigate shared molecular and cellular mechanisms in retinopathies and optic neuropathies and to test neuroprotective interventions.

The administration was carried out via intranasal (IN) route once a day for 14 days after crush (dac). The administration volume was 15 μL/nostril.

The readouts were taken at 14 days after crush.

The neuroprotection was assessed by evaluating RGC survival. Specifically, an IF-confocal analysis for RGC markers (RBPMS) was carried out and activated Caspase3 was assessed to evaluate apoptosis.

The rats were divided into the following groups:
1. Untreated group (Control)—No ONC, no treatment. Rats in this group were not subjected to Optic Nerve Crush and did not receive any treatment.
2. ONC+Vehicle (IN administration). Rats in this group were subjected to Optic Nerve crush and received intranasal vehicle administration.
3. ONC+IN rhNGF 1 (Total administration/day: 9 μg/animal). Rats in this group were subjected to Optic Nerve Crush and received intranasal rhNGF administration. rhNGF concentration for single administration 0.3 mg/mL. Administration volume: 15 μL/nostril—IN treatment starting 4-6 hours after ONC.
4. ONC+IN rhNGF 2 (Total administration/day: 60 μg/animal). Rats in this group were subjected to Optic Nerve Crush and received intranasal rhNGF administration. rhNGF concentration for single administration: 2.14 mg/mL. Administration volume: 15 μL/nostril—IN treatment starting 4-6 hours after ONC.

### Methods

Rats received an overdose of anesthetics on day 14 after ONC (4-6 hours after the treatment). The eyes and optic nerves were removed, post-fixed for 24 h, and dehydrated in 10-30% sucrose solutions. Retinas were dissected and used for flat-mount preparation and analyses of RGC survival.

All immune-reacted sections were analyzed in detail and representative images were performed with the laser-scanning confocal microscope (Olympus FV1200) using a 20× and 40× air and 60× water immersion lens and visualized with FV10-ASW software (Version 4.2, Olympus). An UV diode laser operating at 405 nm, an Argon laser at 488 nm, and a HeNe laser at 543 nm were used as excitation sources. The images were then composed into figures using Adobe illustrator 27.4 and Photoshop CS6.

To quantify RBPMS and Activated Caspase-3 immunoreactivity, 3 (40× magnification) images were captured for each rat (n°3 per group). During the image acquisitions, the exposure parameters, such as gain and time, were kept constant, to avoid observing differences among experimental groups due to artifacts. The analysis of IF staining (% Area RBPMS+ or Activated Caspase-3+) was performed using the ImageJ software (version 1.53, National Institutes of Health, Bethesda, MD, USA, https://imagej.nih.gov/ij/download.html). Statistical analyses were performed using GraphPad InStat3 for Windows. The % Area RBPMS+ or Activated Caspase-3+ quantification was analyzed using one-way analysis of variance (ANOVA) followed by a Tukey's post hoc test. All results are expressed as mean SD, with n being the number of independent experiments.

### Results

The eyes with the nerve were dissected to verify the crush, which was localized within 1 mm from the optic nerve head in all the rat included in the study. The nerve was then dissected from the eye bulb and the entire bulb was processed for slice cut by cryostat. The eye sections were stained with H&E before to be processed for IF analysis.

### Analysis of Activated Caspase-3 in the Retina

The Activated form of Caspase-3 is a reliable marker of cell death and is used in studies of neurodegenerative diseases and other conditions involving cell death, including RGC degeneration following ONC. The expression of activate Caspase-3 in the rat retina following ONC and IN treatments is showed in FIG. 2.

Compared to CTRL, and ONC+rhNGF groups, an intense immunoreactivity for Activate Caspase-3 was found in retina of ONC+Vehicle. The distribution of Activate Caspase-3 was clearly visible in the Ganglion Cell Layer (GCL) where it is expressed by RGCs, but an intense immunoreactivity was also observable in the Inner Nuclear Layer (INL) suggesting that other cell types might also be affected at 14 dac by ONC.

As can be seen in FIG. 2, which shows the evaluation of the % area of Activate Caspase-3 in the different rat groups, the statistical analysis confirmed the activation of apoptotic program in the retina of ONC rats receiving vehicle, and the survival/neuroprotective effects of the intranasal administration of rhNGF at both doses.

### Expression of RBPMS Marker in Retina Following ONC

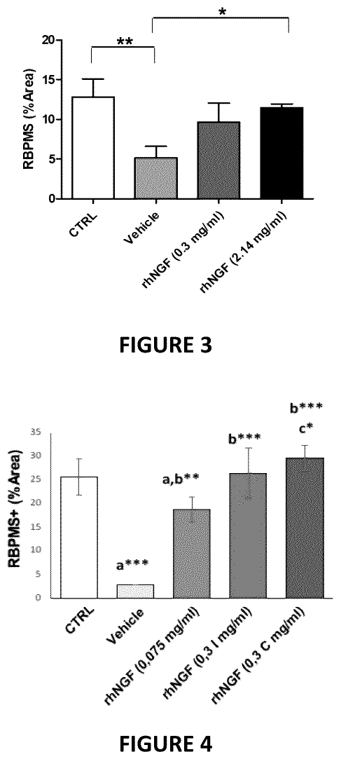

The RBPMS markers is a well characterized RGC marker, which can be used alone to identify the soma of these cells in mammal retina. In line with the expression of Activated Caspase-3, a different distribution of RBPMS marker in GCL of the retina of ONC+Vehicle was observed. The % RBPMS+ area in ONC+Vehicle group resulted decreased compared to CTRL, while an increase in % RBPMS+ area was observed in the ONC+rhNGF groups compared to Vehicle FIG. 3.

To summarize, the intranasal administration of rhNGF in an ONC rat model was found to exert protective effects towards RGCs, as demonstrated by an increase in RBPMS and by a decrease in activated Caspase-3 compared to vehicle-treated animals. This further confirms that, when administered intranasally, rhNGF reaches the optic nerve and the retina at therapeutically effective concentrations and thus exerts a therapeutic effect in retinopathies and optic nerve disorders. There is strengthened support for therapeutic effect in neurodegenerative disorders of the optic nerve, with a possible neuroprotective effect on progressive neuronal degeneration in optic nerve disorders.

### Example 6

A further study was carried out in the same rat model of Optic Nerve Crush (ONC) disclosed in Example 5 to evaluate the neuroprotection of rhNGF intranasal treatment with two different doses and administration schedules.

The administration was carried out via intranasal (IN) route until 14 days after crush (dac).

The administration volume was 15 μL/nostril.

The readouts were taken at 14 days after crush.

The neuroprotection was assessed by evaluating RGC survival. Specifically, an IF-confocal analysis for RGC markers (RBPMS and Tuj1) was carried out.

The rats were divided into the following groups:
1. Untreated group (Control)—No ONC, no treatment. Rats in this group were not subjected to Optic Nerve Crush and did not receive any treatment.
2. ONC+Vehicle (IN administration). Rats in this group were subjected to Optic Nerve crush and received intranasal vehicle administration.
3. ONC+IN rhNGF 0.075 mg/mL (Total administration/day: 2.25 μg/animal). Rats in this group were subjected to Optic Nerve Crush and received intranasal rhNGF administration. rhNGF concentration for single administration: 0.075 mg/mL. Administration volume: 15 μL/nostril—IN treatment starting 3-4 hours after ONC.
4. ONC+IN rhNGF 0.3 mg/ml C (Total administration/day: 9 μg/animal). Rats in this group were subjected to Optic Nerve Crush and received daily intranasal rhNGF administration (continuous administration). rhNGF concentration for single administration: 0.3 mg/mL. Administration volume: 15 μL/nostril—IN treatment starting 3-4 hours after ONC.
5. ONC+IN rhNGF 0.3 mg/mL I (Total administration/day: 9 μg/animal). Rats in this group were subjected to Optic Nerve Crush and received intranasal rhNGF administration with a schedule of treatment of 2 days ON/2 days OFF (intermittent administration). rhNGF concentration for single administration: 0.3 mg/mL. Administration volume: 15 μL/nostril-IN treatment starting 3-4 hours after ONC.

### Methods

Rats received an overdose of anesthetics on day 14 after ONC (4-6 hours after the treatment). The eyes and optic nerves were removed, post-fixed for 24 h, and dehydrated in 10-30% sucrose solutions. Retinas were dissected and used for flat-mount preparation and analyses of RGC survival.

All immune-reacted sections were analyzed in detail and representative images were performed with the laser-scanning confocal microscope (Olympus FV1200) using a 20× and 40× air and 60× water immersion lens and visualized with FV10-ASW software (Version 4.2, Olympus). An UV diode laser operating at 405 nm, an Argon laser at 488 nm, and a HeNe laser at 543 nm were used as excitation sources. The images were then composed into figures using Adobe illustrator 27.4 and Photoshop CS6.

To quantify RBPMS and Tuj1 immunoreactivity, the images were acquired by the laser-scanning confocal microscope (Olympus FV1200) using a 60× water immersion lens, and visualized with FV10-ASW software (Version 4.2, Olympus). An UV diode laser operating at 405 nm, an Argon laser at 488 nm, and a HeNe laser at 543 nm were used as excitation sources. The images were then composed into figures using Adobe illustrator 27.4 and Photoshop CS6. To quantify RBPMS and TuJ1 immuno-reactivity, 3 images were captured for each rat (n=3 per group).

During image acquisitions, the exposure parameters, such as gain and time, were kept constant, to avoid observing differences among experimental groups due to artifacts. The analysis of IF staining (% area) was performed using ImageJ software (version 1.53, National Institutes of Health, Bethesda, MD, USA, https://imagej.nih.gov/ij/download.html). Statistical analyses were performed using GraphPad InStat3 for Windows. The % area quantification was analysed using one-way analysis of variance (ANOVA) followed by a Tukey's post hoc test. All results are expressed as mean±SD (Standard Deviation).

RBPMS+ cells were identified and counted using a semiautomatic method by fixing the common image threshold (the levels were fixed taking the very low IF in ONC retina into account), and cells count was done on digitalizing images using a max white level to identify the IF surrounding nuclei.

### Results

The eyes with the nerve were dissected to verify the crush, which was localized within 1 mm from the optic nerve head in all the rat included in the study. The nerve was then dissected from the eye bulb, and the entire bulb was processed for slice cut by cryostat. The eye sections were stained with H&E before to be processed for IF analysis.

### Expression of RBPMS Marker in Retina Following ONC

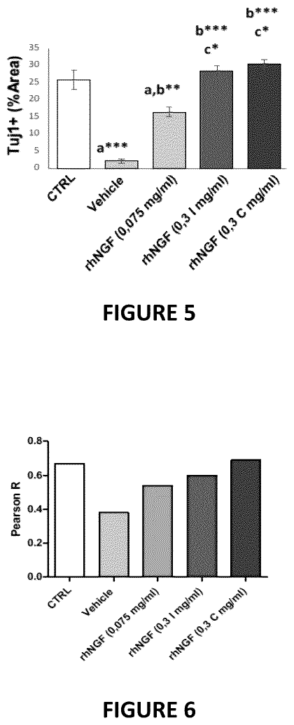

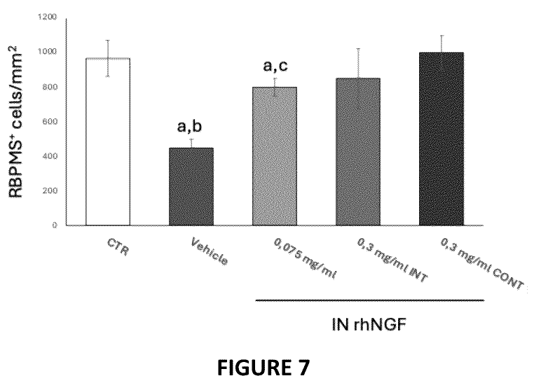

IF analysis confirmed the ONC-induced degeneration of RGCs with significant reduction of both RBPMS (CTRL vs. ONC+Vehicle **p<0.01) and Tuj1 markers (CTRL vs. ONC+Vehicle **p<0.01), and of their related co-expression, in the retina RGC layer of rats that underwent ONC+IN treatments with vehicle when compared to healthy CTRL. In the experimental group treated with the lowest intranasal dose of rhNGF formulation (0.075 mg/ml), a recovery of RBPMS expression (**p<0.01) and an increase of Tuj1 detection (***p<0.001) were observed when compared to ONC+Vehicle. A marked increase of RGC expressing RBPMS and Tuj1 was found also in the retina of ONC rats receiving intranasal treatment with 0.3 mg/ml of rhNGF for 14 days following ONC, both with a daily continuous treatment and with an intermittent treatment schedule (***p<0.001) (FIGS. 4 and 5). As can be seen from FIG. 6, the Pearson's R graph indicates the degree of colocalization between the two RGC markers, RBPMS and Tuj1, further strengthening the validity of the data. In line with these results, as can be seen from FIG. 7, the number of RBPMS+ was lower in the ONC group treated with intranasal administration of vehicle compared to control group. Conversely, the number of RBPMS+ cells was increased in all ONC groups treated with intranasal rhNGF formulations in a dose-dependent manner, being comparable to control for the ONC group treated with continuous administration of 0.3 mg/ml of rhNGF.

To summarize, the intranasal administration of different doses with different administration schedules of rhNGF in an ONC rat model was found to exert protective effects towards RGCs, as demonstrated by an increase in RBPMS and Tuj1 compared to vehicle-treated animals. This confirms that, when administered intranasally, rhNGF reaches the optic nerve and the retina at therapeutically effective concentrations and thus exerts a therapeutic effect in retinopathies and optic nerve disorders. There is strengthened support for therapeutic effect in neurodegenerative disorders of the optic nerve, with a possible neuroprotective effect on progressive neuronal degeneration in optic nerve disorders.

### Example 7

A study is performed to evaluate the Efficacy and Safety of Intranasal Recombinant Human Nerve Growth Factor (rhNGF) (Cenegermin) in Adult Participants with Non-Arteritic Anterior Ischemic Optic Neuropathy (NAION).

Objectives and endpoints

| Objective | Endpoints |
|---|---|
| Primary | |
| To evaluate the | Improvement in Best Corrected Visual |
| efficacy of intranasal | Acuity (BCVA), defined as a ≥15 letter |
| cenegermin compared | gain from baseline to Week 24, measured |
| to vehicle on | using the Early Treatment Diabetic |
| improving the visual | Retinopathy Study (ETDRS) chart. |
| acuity in participants | |
| with NAION. | |
| Key secondary | |
| To evaluate the | Change from baseline through Week 24 |
| efficacy over time of | in visual field mean sensitivity in decibels. |
| intranasal cenegermin | Change from baseline through Week 24 |
| compared to | in BCVA as measured by the ETDRS |
| vehicle on improving | chart. |
| the visual function | Change from baseline through Week 24 |
| and structural changes | in visual field mean sensitivity, in a |
| in the ganglion cell | prespecified region consisting of at least 5 |
| layer in participants | separate loci on visual field testing. |
| with NAION. | Change from baseline through Week 24 |
| | in ganglion cell layer-inner plexiform layer |
| | (GCL-IPL) thickness (um) on optical |
| | coherence tomography (OCT). |
| Secondary | |
| To evaluate the | Improvement in BCVA, defined as a ≥10 |
| efficacy over time of | letter gain from baseline through Week 24, |
| intranasal cenegermin | measured by the ETDRS chart. |
| compared to | Worsening in BCVA, defined as a ≥15 |
| vehicle on improving | letter loss from baseline through Week 24, |
| the visual acuity in | measured by the ETDRS chart. |
| participants with | Worsening in BCVA, defined as a ≥10 |
| NAION. | letter loss from baseline through Week 24, |
| | measured by the ETDRS chart. |
| To evaluate the | Improvement in visual field, defined as a |
| efficacy over time of | gain of ≥7 decibels from baseline to Week |
| intranasal cenegermin | 24 in mean sensitivity on visual field. |
| compared to | Improvement in visual field, defined as a |
| vehicle on improving | gain of ≥7 decibels from baseline to Week |
| the visual field in | 24 in a prespecified region consisting of at |
| participants with | least 5 separate loci on visual field testing. |
| NAION. | Worsening in visual field, defined as a |
| | loss of ≥7 decibels from baseline to Week |
| | 24 in mean sensitivity on visual field. |
| | Worsening in visual field, defined as a |
| | loss of ≥7 decibels from baseline to Week |
| | 24 in a prespecified region consisting of at |
| | least 5 separate loci on visual field testing. |
| To evaluate the | Change from baseline through Week 24 |
| efficacy of intranasal | in the following OCT parameters, which |
| cenegermin compared | may include but are not limited to: |
| to vehicle on the | i) Thickness (um) of Retinal Nerve Fiber |
| structural changes of | Layer (RNFL) |
| optic nerve head and | ii) Thickness (um) of the Ganglion Cell |
| retina. | Layer (GCL), Ganglion Cell Complex |
| | (GCC), or GCL-IPL |
| | Time to resolution of optic disc edema as |
| | follows: |
| | i) Time to resolution (days) of the optic disc |
| | edema as measured by investigator |
| | clinical exam or fundus photography, with |
| | symptom onset as baseline |
| | ii) Time to resolution (days) of the optic |
| | disc edema as measured by investigator |
| | clinical exam or fundus photography, with |
| | treatment initiation as baseline |
| | iii) Time to onset (days) of optic disc |
| | atrophy as measured by investigator |
| | clinical exam or fundus photography, with |
| | symptom onset as baseline |
| | iv) Time to onset (days) of optic disc |
| | atrophy as measured by investigator |
| | clinical exam or fundus photography, with |
| | treatment initiation as baseline. |
| To evaluate the | Change from baseline through Week 24 in |
| effect of intranasal | the National Eye Institute Visual Function |
| cenegermin compared | Questionnaire (NEI-VFQ-25) score. |
| to vehicle on | |
| vision-related quality | |
| of life (VR-QoL). | |
| Exploratory | |
| To evaluate the | Improvement in visual function, defined |
| efficacy of intranasal | as a gain of ≥15 letters from baseline to |
| cenegermin compared | Week 24 in BCVA measured by the |
| to vehicle on | ETDRS chart and/or a gain of ≥7 decibels |
| improving the overall | in a prespecified region consisting of at |
| visual function in | least 5 separate loci on visual field testing |
| participants | Worsening in visual function, defined as |
| with NAION. | a loss of ≥15 letters from baseline to Week |
| | 24 in BCVA measured by the ETDRS |
| | chart and/or a loss of ≥7 decibels in a |
| | prespecified region consisting of at least 5 |
| | separate loci on visual field testing. |
| | Change in Quantitative Contrast |
| | Sensitivity Function (qCSF)a (which may |
| | also include quantitative visual acuity) |
| | from baseline through Week 24, as |
| | measured by Manifold device. |
| | Change in BCVA (letters) from nadir to |
| | BCVA measured at Week 24. |
| | Change in visual field mean sensitivity |
| | (decibels) from nadir to mean sensitivity |
| | measured at Week 24. |
| To evaluate the | Change from baseline through Week 24 in |
| efficacy of intranasal | parameters assessed by OCT |
| cenegermin compared | angiography (OCTA)a , which may include |
| to vehicle on the | but are not limited to: |
| structural changes of | Flow impairment of radial peripapillary |
| the optic nerve head, | capillaries |
| retina, and | Flow impairment of peripapillary |
| microvasculature. | choriocapillaris. |

Abbreviations: BCVA = best corrected visual acuity; ETDRS = Early Treatment Diabetic Retinopathy Study chart; GAT = Goldmann applanation tonometry; GCL-IPL = ganglion cell layer-inner plexiform layer; IOP = increased intraocular pressure; NAION = Non-arteritic anterior ischemic optic neuropathy; NEI-VFQ-25 = National Eye Institute Visual Function Questionnaire-25; NGF = nerve growth factor; OCT = optical coherence tomography; OCTA = optical coherence tomography angiography; PSD = pattern standard deviation; qCSF = quantitative contrast sensitivity function; RAPD = relative afferent pupillary defect; RNFL = retinal nerve fiber layer; VFI = visual field index; VR-QoL = vision-related quality of life.

Population: individuals aged 50 to 80 years with an eye within 14 days of symptom onset of NAION, with BCVA score of ≥15 letters and ≤65 letters measured using the ETDRS chart and satisfying all other inclusion/exclusion criteria.

Participants who sign informed consent will undergo screening (visit 1) to determine study eligibility for enrolment. Approximately 272 participants who continue to meet all eligibility criteria at baseline (day 1) will be randomized in a 1:1 ratio to receive either 4 cycles of cenegermin (recombinant human nerve growth factor having the amino sequence of SEQ ID NO: 1) treatment (group 1) or vehicle control (group 2). Participants will be stratified according to 2 stratification factors: number of days since symptom onset (0 to 7 days versus 8 to 14 days) and BCVA letter score at screening (15 to 49 letters versus 50 to 65 letters). Each of the 4 treatment cycles will last 14 days, consisting of a treatment period of 7 consecutive days of investigational medicinal product (IMP) administration once daily, followed by a 7-day period during which no IMP will be administered.

Treatment will be administered in a masked fashion across parallel arms for 8 weeks as follows:
Group 1 (cenegermin): Approximately 136 participants will receive 4 treatment cycles of cenegermin 180 μg
Group 2 (vehicle): Approximately 136 participants will receive 4 treatment cycles of vehicle. Participants randomized to the vehicle group will receive the same volume and number of sprays as those randomized to the cenegermin group via the same mucosal atomization device. Aside from the absence of cenegermin drug substance, the vehicle is identical to drug product and has the same container closure system, dosage form, and preparation methods.

After this 8-week treatment period, participants will be followed for another 16 weeks in a follow-up period for efficacy and safety assessments.

Participants will be enrolled within 14 days of symptom onset to capture the acute pathogenic phase of disease, where cenegermin may exhibit the greatest neuroprotective impact by potentially mitigating ongoing oxidative damage.

### Inclusion Criteria

Participants are eligible to be included in the study only if all of the following criteria apply:
1. Participant must be 50 to 80 years of age inclusive, at the time of signing informed consent.
2. A clinical diagnosis of unilateral NAION in the study eye with symptom onset within 14 days prior to the planned date for first dose administration, confirmed with all the following at screening visit:
a. ONH edema
b. Visual field pattern compatible with the diagnosis of NAION, confirmed by central reading center (CRC) assessment
c. Visual field defects with mean sensitivity ≥7 decibels and ≤30 decibels
d. Optical coherence tomography (OCT) findings consistent with a diagnosis of NAION, according to CRC assessments
e. Relative afferent pupillary defect in the study eye (not required if the fellow eye had previous NAION or other optic nerve or retinal disease that is not an exclusion criterion).
3. Participant must be eligible for randomization and receive the first dose within 14 days of symptom onset.
4. A BCVA score in the study eye of ≥15 letters and ≤65 letters measured using the ETDRS chart.
5. Sufficiently clear ocular media and adequate pupil dilation to enable assessment of the optic nerve and retina in both eyes.

### Exclusion Criteria

Participants are excluded from the study if they have any of the following criteria:
1. Bilateral NAION or sequential NAION with fellow eye involvement within 6 weeks of study eye involvement.
2. Clinical evidence of temporal arteritis (giant cell arteritis) signs or symptoms, where any of the following applies:
a. jaw claudication
b. scalp tenderness
c. temple tenderness overlying the temporal arteries
d. pallid disc edema
e. Two or more of the following: previous episodes of transient visual loss leading up to persistent visual loss, headache, proximal myalgias, anorexia, weight loss, fever
3. Abnormal laboratory findings suggestive of temporal arteritis (giant cell arteritis), in the absence of a known acute cause (eg, in absence of infection, anemia, trauma):
a. C reactive protein (CRP) level >2× the institutional upper limit of normal (ULN) OR b. Elevated erythrocyte sedimentation rate (ESR), defined as >age/2 mm/hr for males or > (age+10)/2 mm/hr for females OR
c. Thrombocytosis, defined as a platelet count greater than >450,000 μl on complete blood count.
4. Pain with eye movement
5. Hemoglobin level less than 10 g/dL 6. Intraocular pressure (IOP) greater than 25 mmHg in the study eye or history of glaucoma in the study eye.
7. Intermediate age-related macular degeneration (AMD) with subfoveal drusen, exudative AMD, or geographic atrophy in the study eye.
8. Uncontrolled diabetes mellitus (HbA1c≥8.0%), any level of diabetic retinopathy, or previous pan-retinal laser photocoagulation or macular laser photocoagulation.
9. History or evidence of:
a. Optic neuritis or intraocular inflammation (chronic or recurrent anterior uveitis, any intermediate uveitis, or any posterior uveitis) in either eye.
b. Infectious, nutritional, hereditary, radiation-induced, neoplastic (tumor-related), toxic and mitochondrial optic neuropathies, or any active ocular infection in either eye.
c. Multiple sclerosis, collagen vascular disease, other systemic chronic inflammatory disease, or chronic immunosuppressive disease (such as HIV/AIDS).
10. Amblyopia or any other potential cause of vision loss in study eye not associated with NAION.
11. Malignancy, defined as any of the following:
a. Known or suspected ocular malignancy (eg, ocular surface, intraocular, ocular adnexa)
b. Presence of malignancy or any other systemic disease that, in the opinion of the investigator, may interfere with the participant's ability to comply with or complete the clinical study, including basal cell carcinoma of the head
c. History of malignancy, as follows: i. Malignancy within the past 5 years requiring oncologic treatment (eg, surgery, chemotherapy, immunotherapy, or radiotherapy), except for non-facial basal cell carcinoma that has been adequately treated ii. Any history of CNS malignancy
12. Prior use or planned use during the study of any of the following prior to informed consent:
a. Systemic corticosteroids within 1 month
b. Intranasal drug products for more than 7 cumulative days within 1 month
c. Any medication or supplement with presumed neuroprotective effect (eg, systemic nicotinamide, systemic memantine, topical apraclonidine, topical brimonidine) within 6 weeks
d. Immunomodulatory therapy within 3 months
e. Amiodarone within 12 months
f. Chemotherapy within 3 years
g. Radiation therapy of head or neck within 20 years
h. Any history of medication or supplement with known risk of other optic neuropathy or retinal toxicity (eg, ethambutol, hydroxychloroquine, chloroquine)
13. Planned use of phosphodiesterase-5 (PDE-5) inhibitors during the study
14. Conditions that prevent the effective intranasal delivery of IMP:
a. known history of nasal polyps, chronic sinusitis, anatomical abnormalities obstructing the nasal passages
b. prior history of paranasal or endoscopic nasal procedures (eg, paranasal sinus surgery, endoscopic nasal surgery such as transsphenoidal resection of pituitary tumors)
15. History of or known active substance abuse or dependency, including but not limited to alcohol, illicit drugs, misuse of prescription medications; marijuana or cannabis-derived products.
16. Affirmative response to questions 4 or 5 of the Columbia Suicidality Severity Rating Scale (C-SSRS) at screening.
17. Participants who have enrolled in another ophthalmological clinical trial within 2 months prior to informed consent or who are scheduled to enrol in another clinical trial during this study.
18. History of adverse reactions or significant hypersensitivity to any drug or excipient used for the study.
19. Presence or history of any ocular or systemic disease or condition that may affect the efficacy or evaluation of study treatment, interfere with the interpretation of study results, or be considered by the investigator to be incompatible with the study visit schedule or study conduct.

### Efficacy Assessment

### Best Corrected Visual Acuity (Early Treatment Diabetic Retinopathy Study Chart)

Best Corrected Visual Acuity (BCVA) will be assessed using the Early Treatment Diabetic Retinopathy Study (ETDRS) letter chart at a starting test distance of 4.0 meters under standardized lighting conditions (Beck et al, 2007). The ETDRS letter score has a maximum of 100, with a higher score indicative of better visual acuity. BCVA testing should be performed prior to the administration of any dilating eye drops or any examination that requires contact with the eye.

At the screening, baseline, Week 24 (EOS), and early discontinuation visits, BCVA should be performed 2 or 3 times in the study eye.
If there is a difference of 5 letters or fewer between the first 2 BCVA measurements, the average of these 2 measurements will be calculated and recorded as the BCVA for this timepoint. A third measurement will not be required.
If the difference between the first 2 measurements is greater than 5 letters, a third measurement will be taken.
If the third measurement differs by 5 letters or fewer from the second measurement, the average of the second and third measurements will be recorded as the BCVA for this timepoint.
If the third measurement differs by 5 letters or fewer from the first measurement, the average of the first and third measurements will be recorded as the BCVA for this timepoint.
If the third measurement differs by 5 letters or fewer from both the first and second measurements, the average of all 3 measurements will be recorded as the BCVA for this timepoint.
If the third measurement differs by more than 5 letters from both the first and second measurements, the participant will be considered ineligible.

### Visual Field Testing

Visual field testing will be conducted to assess visual function and monitor ocular safety using validated automated perimetry (Vesti et al, 2003). At baseline visit and Week 24 (EOS), full-threshold perimetry using spot size V (SSV) will be performed twice in the study eye, and both assessments will be sent to the CRC for evaluation. Visual field testing will be performed in each eye (the study eye and fellow eye separately) at the screening visit, baseline visit, Week 8, Week 24 (EOS), and early discontinuation visit.

### Quantitative Contrast Sensitivity Function Assessment

Quantitative Contrast Sensitivity Function (qCSF) is a computerized, adaptive psychophysical test used to evaluate a participant's contrast sensitivity across a large range of spatial frequencies (Vingopoulos et al, 2021). This will be conducted using the Manifold device which contains the Bayesian adaptive algorithm to present a series of optotypes (letters) that vary simultaneously in spatial frequency and contrast, allowing for rapid estimation of the participant's full contrast sensitivity function. Subtle changes in central vision that are not detected by BCVA assessment can be detected more sensitively by contrast sensitivity assessment. qCSF is an optional exploratory assessment and will be conducted only at sites with the capabilities to perform it.

### National Eye Institute Visual Functioning Questionnaire-25

The National Eye Institute Visual Functioning Questionnaire-25 (NEI-VFQ-25) is a validated, patient-reported outcome tool developed to measure the impact of visual impairment on quality of life (Mangione et al, 2001). It consists of 25 core items across 12 subscales of visual function: general health, general vision, ocular pain, near and distance activities, social functioning, mental health, role difficulties, dependency, driving, color vision, and peripheral vision. The numeric responses are converted to a scale ranging from 0 to 100, where higher scores indicate better vision-related functioning and quality of life. Items within each subscale (excluding the single general health question) are averaged to generate a composite score (maximum of 100).

### Optical Coherence Tomography

Optical coherence tomography (OCT) is a non-invasive imaging technique that provides high-resolution, cross-sectional images of microstructure within tissues by using measurements of optical backscattering (Fujimoto et al, 2000). In this study, OCT will be used to explore potential structural changes in the optic nerve head, retina, and microvasculature by measuring various parameters (eg, thickness of the retinal nerve fiber layer [RNFL], ganglion cell layer [GCL], ganglion cell complex [GCC], ganglion cell layer-inner plexiform layer [GCL-IPL], time to resolution of optic disc edema).

### Optical Coherence Tomography Angiography

OCT Angiography (OCTA) is a non-invasive imaging modality that enables high-resolution visualization of the retinal and choroidal vasculature without the use of dye, by detecting motion contrast from RBC movement (Le et al, 2025). OCTA will be used to assess exploratory efficacy by measuring various parameters over time such as flow impairment of radial peripapillary capillaries and peripapillary choriocapillaris. OCTA is an optional exploratory assessment and will be conducted only at sites with the capabilities to perform it.

### Fundus Photography

Fundus photography uses a non-invasive camera with a bright flash to take photographs of the retina (Panwar et al, 2016). It supports evaluation of the optic nerve, and it will be used to monitor safety as well as efficacy.

### Intraocular Pressure (IOP)

Intraocular pressure (IOP) will be measured by tonometry (eg, Goldmann applanation tonometry, handheld tonometer) for each eye to monitor ocular safety and tolerability (Asrani et al, 2024). The measurement will be performed following instillation of a topical anesthetic and fluorescein dye.

### Slit Lamp Examination

Slit lamp examination will be performed during every visit to assess ocular safety and monitor any abnormalities in the anterior and posterior segments of the eye (Martin 2018). The anterior segment examination will assess the eyelids and eyelashes, conjunctiva, cornea, anterior chamber, iris and pupil, lens, and anterior vitreous. The posterior segment lens will be used to evaluate the vitreous, optic nerve head, and the retina and macula.

### Dilated Fundus Examination

After specular microscopy, mydriatic drops will be instilled in both eyes and once adequate dilation is achieved, a fundal exam shall be performed to examine the vitreous, retina, and optic nerve (Upadhyaya et al, 2022). Features of interest will be vitritis, vitreal or retinal hemorrhage, maculopathy, retinal tears or detachment, posterior vitreous detachment, altered optic nerve appearance, and optic nerve cup-to-disc ratio.

### Relative Afferent Pupillary Defect

Relative afferent pupillary defect (RAPD) is a standard eye assessment performed by shining a bright light (typically a penlight or flashlight) into one eye followed by the fellow eye (Broadway 2012). The swinging flashlight test can elicit a RAPD. Presence of RAPD would indicate that there is significant optic nerve damage to one eye relative to its fellow eye. If both eyes have significant optic nerve damage, then the RAPD test would be negative despite underlying optic nerve disease.

1. A method for treating a human subject for non-arteritic anterior ischemic optic neuropathy (NAION) comprising intranasally administering human nerve growth factor.

2. The method of claim 1, wherein the human nerve growth factor is administered to the human subject daily for at least one week.

3. The method of claim 2, wherein the human nerve growth factor is administered to the human subject by intermittent administration for at least 3 cycles, wherein each cycle comprises one week of daily administration with human nerve growth factor followed by one week without administration of human nerve growth factor.

4. The method of claim 2, wherein the human nerve growth factor is administered to the human subject by intermittent administration for 4 cycles, wherein each cycle comprises one week of daily administration with human nerve growth factor followed by one week without administration of human nerve growth factor.

5. The method of claim 2, wherein the daily administration of human nerve growth factor comprises one intranasal administration of human nerve growth factor per day.

6. The method of claim 2, wherein the amount of human nerve growth factor administered per daily administration is between 15 μg and 2100 μg.

7. The method of claim 2, wherein the amount of human nerve growth factor administered per daily administration is between 30 μg and 1200 μg.

8. The method of claim 2, wherein the amount of human nerve growth factor administered per daily administration is between 100 μg and 300 μg.

9. The method of claim 2, wherein the amount of human nerve growth factor administered per daily administration is about 180 μg.

10. The method of claim 1, wherein the human nerve growth factor is recombinant human nerve growth factor.

11. The method of claim 3, wherein the amount of human nerve growth factor administered per daily administration is between 15 μg and 2100 μg.

12. The method of claim 1, wherein the human nerve growth factor has the amino acid sequence of (SEQ. ID NO. 1), (SEQ ID NO: 2); or is a mixture of SEQ ID NO. 1 and SEQ ID NO. 2.

13. The method of claim 1, wherein the intranasal administration comprises intranasally administering a liquid pharmaceutical composition comprising human nerve growth factor in an amount between 80 μg/ml and 240 μg/ml.

14. The method of claim 1, wherein the intranasal administration comprises intranasally administering a liquid pharmaceutical composition comprising about 180 μg/ml of human nerve growth factor.

15. The method of claim 13, wherein the liquid pharmaceutical composition is administered to the human subject daily for at least one week.

16. The method of claim 13, wherein the liquid pharmaceutical composition is administered to the human subject by intermittent administration for at least 3 cycles, wherein each cycle comprises one week of daily administration followed by one week without administration of the liquid pharmaceutical composition.

17. The method of claim 13, wherein the liquid pharmaceutical composition is administered to the human subject by intermittent administration for 4 cycles, wherein each cycle comprises one week of daily administration followed by one week without administration of the liquid pharmaceutical composition.

18. The method of claim 13, wherein the daily administration of liquid pharmaceutical composition comprises one intranasal administration per day.

19. The method of claim 13, wherein the amount of human nerve growth factor administered per daily administration is between 15 μg and 2100 μg.

20. The method of claim 13, wherein the amount of human nerve growth factor administered per daily administration is between 30 μg and 1200 μg.

21. The method of claim 13, wherein the amount of human nerve growth factor administered per daily administration is between 100 μg and 300 μg.

22. The method of claim 13, wherein the amount of human nerve growth factor administered per daily administration is about 180 μg.

23. The method of claim 13, wherein the human nerve growth factor is recombinant human nerve growth factor.

24. The method of claim 13, wherein the human nerve growth factor has the amino acid sequence of (SEQ. ID NO. 1), (SEQ ID NO: 2); or is a mixture of SEQ ID NO. 1 and SEQ ID NO. 2.

25. The method of claim 13, wherein the human nerve growth factor is the only active ingredient in the pharmaceutical composition.

26. The method of claim 13, wherein the pharmaceutical composition does not comprise one or more of: brain derived neurotrophic growth factor (BDNF), transforming growth factor, epidermal growth factor (EGF), platelet derived growth factor (PDGF), and an interleukin.

27. The method of claim 16, wherein the pharmaceutical composition does not comprise any of: brain derived neurotrophic growth factor (BDNF), transforming growth factor, epidermal growth factor (EGF), platelet derived growth factor (PDGF), and an interleukin.


SEQUENCE LISTING (6 sequences)

SEQ ID NO: 1 - protein, 118 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 2 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - protein, 118 aa, synthetic construct
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWEFIRIDTACVCVLSRKAVR

SEQ ID NO: 4 - protein, 120 aa, synthetic construct
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWEFIRIDTACVCVLSRKAVRRA

SEQ ID NO: 5 - protein, 118 aa, synthetic construct
SSSHPIFHRGEFSVCDSVSVWVGDKTTATDIKGKEVMVLGEVNINNSVFKQYFFETKCRDSNPVDSGCRGIDSKHWNSYCTTTHTFVKALTMDGKQAAWEFIRIDTACVCVLSRKAVR

SEQ ID NO: 6 - protein, 120 aa, synthetic construct
SSSHPIFHRGEFSVCDSVSVWVGDKTTATDIKGKEVMVLGEVNINNSVFKQYFFETKCRDSNPVDSGCRGIDSKHWNSYCTTTHTFVKALTMDGKQAAWEFIRIDTACVCVLSRKAVRRA